(19) United States
(12) Patent Application Publication
(10) Pub. No.: US 2010/0234750 A1
(43) Pub. Date: Sep. 16, 2010

(54) APPARATUS AND METHOD FOR DETECTING BREATHING DISORDERS

(75) Inventors: Arie ARIAV, Doar-Na Hof Ashkelon (IL); Benjamin Veikhman, Rechovot (IL); Vladimir Ravitch, Ashkelon (IL)

Correspondence Address: MARTIN D. MOYNIHAN d/b/a PRTSI, INC., P.O. BOX 16446, ARLINGTON, VA, 22215, US

(73) Assignee: Nexense Ltd., Yavne (IL)

(21) Appl. No.: 12/707,756

(22) Filed: Feb. 18, 2010

Related U.S. Application Data
(60) Provisional application No. 61/153,663, filed on Feb. 19, 2009.

(51) Int. Cl. A61B 5/08

(52) U.S. Cl. 600/534

(57) ABSTRACT

Apparatus for detecting breathing disorders in a person, comprising a sensor constructed to be located on, in, or under a mattress to sense mechanical vibrations of a body part of the person while lying on the mattress, and a control system designed to receive the output of the sensor, and to perform the following operations:
filter out the breath components of the sensor output;
sample the breath components during predetermined short time durations (N_{S});
determine the average energy E_{S} in the samples;
sample breath components during long time durations (N_{L});
determine the average energy (E_{L}) in the latter samples;
calculate a breath quality factor (BQF) by dividing E_{S} by E_{L} (E_{S}/E_{L}); and
actuating a display, alarm, and/or control when the BQF is a predetermined value less than 1.0.

### RELATED APPLICATIONS

This application claims the benefit of priority of U.S. Provisional Patent Application No. 61/153,663, filed on Feb. 19, 2009, the contents of which are incorporated herein by reference.

### FIELD AND BACKGROUND OF THE INVENTION

The present invention relates to an apparatus and a method for detecting breathing disorders. The invention is particularly useful for detecting such breathing disorders as asthma, chronic obstructive pulmonary disease (COPD), sleep apnea, and cystic fibrosis (CF), or other conditions which are indicated by breathing disorders.

Detecting breathing disorders is commonly done in sleep laboratories using a plurality of sensors for detecting respiration, heart activity, movements, and the like. This requires expensive laboratory equipment available only at sleep laboratories, and further requires that the patient spend the night at the sleep laboratory.

PCT Application Nos. PCT/IL2005/000617, published on Dec. 22, 2005 as Publication No. WO2005/120167 and PCT/IL2007/000636, published on Dec. 6, 2007 as Publication No. WO2007/138575, both assigned to the same assignee as the present application, disclose methods and apparatus that can be used for detecting such breathing disorders, particularly snoring, which do not require the expense or inconvenience of a sleep laboratory, and which can be performed at home. Other techniques for detecting such sleep disorders are described in U.S. Pat. Nos. 6,468,234 and 7,077,810. A main problem in the methods and apparatus heretofore used for detecting breathing patterns, both in a sleep laboratory and in the techniques described in the above-cited patent publications, is the dependence on the position of the patient with respect to the sensors to avoid false detections or misdetections of disorders.

### OBJECTS AND BRIEF SUMMARY OF THE PRESENT INVENTION

An object of the present invention is to provide a novel apparatus and method having advantages in the above respects. Another object of the invention is to provide apparatus and a method for detecting breathing disorders operating according to an algorithm which does not depend on patient position.

According to one broad aspect of the present invention, there is provided apparatus for detecting breathing disorders in a person, comprising: a sensor constructed to be located on, in, or under a mattress and to sense mechanical vibrations of a body part of the person while lying on the mattress; and a control system designed to receive the output of the sensor, and to perform the following operations: (a) filter out the breath components of the sensor output; (b) sample the breath components during predetermined short time durations (N_{S}); (c) determine the average energy E_{S} in the samples; (d) sample the breath components during long time durations (N_{L}); (e) determine the average energy (E_{L}) in the latter samples; (f) calculate a breath quality factor (BQF) by dividing E_{S} by E_{L} (E_{S}/E_{L}); and (f) actuating a display, alarm, and/or control when the BQF is a predetermined value less than 1.0.

In the preferred embodiment of the invention described below, in operation (a), the breath components filtered out are of a frequency of 0.05-0.5 Hz. In operation (b) the predetermined short time durations are 3-5 seconds, and in operation (d) said long time durations are 25-30 seconds.

In operations (c) and (e), the average energy of said predetermined short time durations (E_{S}) and said predetermined long time durations (E_{L}) are calculated as follows:

$$E ( k ) = {\sum }_{i - N + 1}^{k} {S}^{2} ( i ) \cdot Δ t$$

Here Δt—signal sampling (about 10 msec);
S(i)—i-th sample of the filtered signal;
k—the number of current sample;
N—the length of sliding window (duration).

According to still further features in the described preferred embodiment, the operation (f) the breath quality factor is calculated as follows:

$$B Q F ( k ) = \frac{{E}_{S} / {N}_{S}}{{E}_{L} / {N}_{L}} ;$$

and in operation (g), the display, alarm and/or control is actuated when the breath quality factor is between 0.5-0.7.

Best results have been obtained when the sensor is an acoustical sensor which includes: an acoustical transmitter; an acoustical receiver spaced from the acoustical transmitter to define an acoustical transmission channel therebetween adapted to be located, with respect to the person, such that the mechanical vibrations of the person's body part change the length of the acoustical transmission channel; and a measuring circuit for measuring the transit time of an acoustical signal transmitted from the acoustical transmitter to the acoustical receiver. Such a sensor is more particularly described in the above-cited published PCT patent applications, the contents of which are herein incorporated by reference.

According to another aspect of the invention, there is provided a method for detecting breathing disorders in a person, comprising: sensing mechanical vibrations of a body part of a person while lying on a mattress; converting the mechanical vibrations to electrical signals; and processing the electrical signals by the following operations: (a) filtering out the breath components of the sensor output; (b) sampling the breath components during predetermined short time durations; (c) determining the average energy E_{S} in the samples; (d) sampling the breath components during long time durations; (e) determining the average energy (E_{L}) in the latter samples; (f) calculating a breath quality factor (BQF) by dividing E_{S} by E_{L} (E_{S}/E_{L}); and (g) actuating a display, alarm, and/or control when the BQF is a predetermined value less than 1.0.

As will be described more particularly below, such apparatus and method may be practiced with relatively simple and inexpensive equipment which does not require the facilities of a sleep laboratory, and which provides robust detection of breathing disorders not dependent on patient position.

Further features and advantages of the invention will be apparent from the description below.

### BRIEF DESCRIPTION OF THE DRAWINGS

The invention is herein described, by way of example only, with reference to the accompanying drawings, wherein:

FIG. 1 diagrammatically illustrates one form of apparatus constructed in accordance with the present invention;

FIG. 2 is a block diagram illustrating the vibration sensor and the control system in the apparatus of FIG. 1;

FIG. 3 is a flow chart illustrating the algorithm used in operating the system of FIGS. 1 and 2 for detecting breathing disorders; and

FIG. 4 are signal wave diagrams helpful in understanding the flow chart of FIG. 3.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF A PREFERRED EMBODIMENT

In the preferred embodiment of the invention as described below, and as illustrated in the accompanying drawings, the sensor apparatus is based on the apparatus described in the above-cited published PCT applications by the applicant of the present invention, but programmed to provide a robust method for detecting breathing disorders which are not dependent on the patient position. Rather, the breathing disorders are detected according to an algorithm based on the calculation of a certain relative parameter, which is called Breath Quality Factor (BQF), whose value does not depend on patient position.

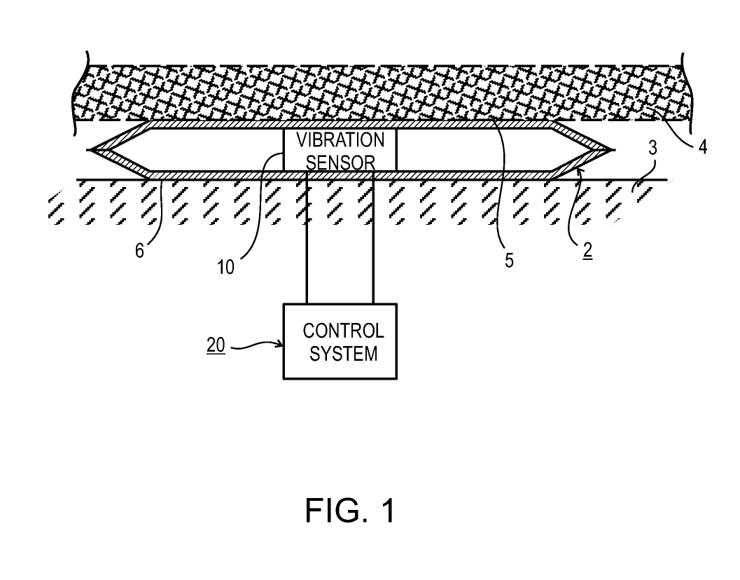

Reference is first made to FIG. 1 illustrating the overall construction of such an apparatus. The apparatus includes a sensor, generally designated 2, constructed to be located on, in, or under a mattress so as to sense mechanical vibrations of a body part of the person while lying on the mattress. In the example illustrated in FIG. 1, sensor 2 is located between the mattress supporting panel 3 and the mattress 4.

Sensor assembly 2 includes a housing defined by an upper plate 5 and a lower plate 6. A vibration sensor unit, generally designated 10, is located centrally between the two plates 5 and 6 such that the opposite faces of the vibration sensor firmly contact the inner surfaces of the two plates, such that it will sense the vibrations of the upper plate 5 with respect to the lower plate 6. The output of vibration sensor 10 is fed to a control system, generally designated 20.

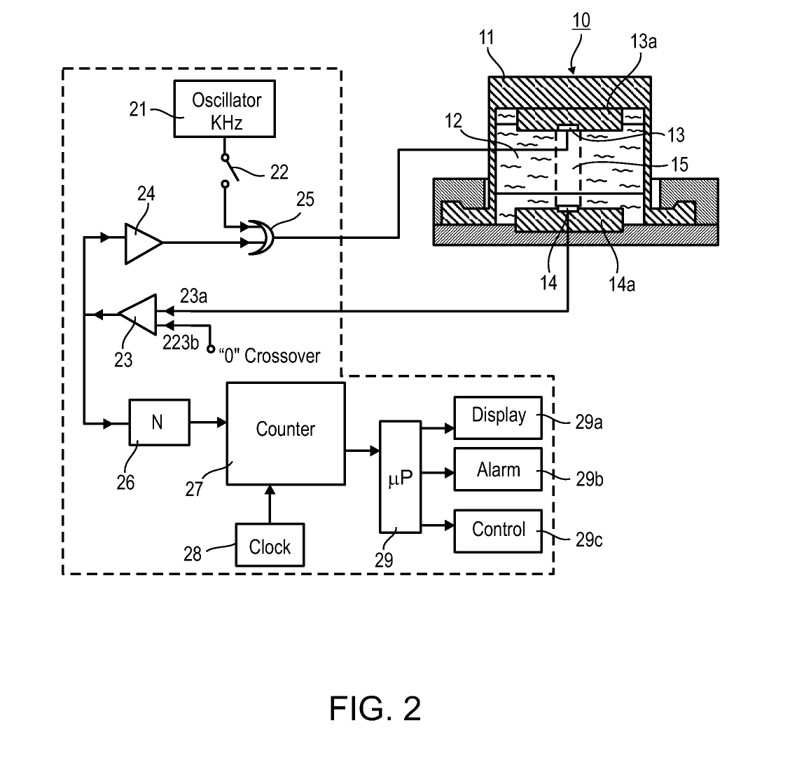

The construction of the sensor 10 and the control system 20 is more particularly illustrated in FIG. 2.

The vibration sensor 10 is one of those described in the above-cited published PCT Application No. PCT/IL2005/000617. Briefly, it includes a housing 11 filled with a liquid 12 having high transmissivity and low attenuation properties with respect to acoustical waves. Preferably, liquid 12 is a silicone oil having high viscosity properties, similar to honey.

Sensor 10 further includes an acoustical transmitter 13 and an acoustical receiver 14 carried on opposed walls of housing 11. The two walls are spaced apart from each other so as to define, between transmitter 13 and receiver 14, an acoustical transmission channel, generally designated 15 constituted of the liquid 12 within the housing. Transmitter 13 and receiver 14 are each carried by damper elements 13a and 14a, respectively, having high attenuation properties with respect to acoustical waves, such that the waves are substantially restricted to channel 15.

Wall 11 of housing 10 is deformable by the vibrations transmitted to it from mattress 4, so that the distance between the transmitter 13 and receiver 14 will vary as a result of such vibrations. Thus, the vibrations can be detected by measuring the transit time of an acoustical wave, transmitted from transmitter 13 to receiver 14, through the acoustical transmission channel 15.

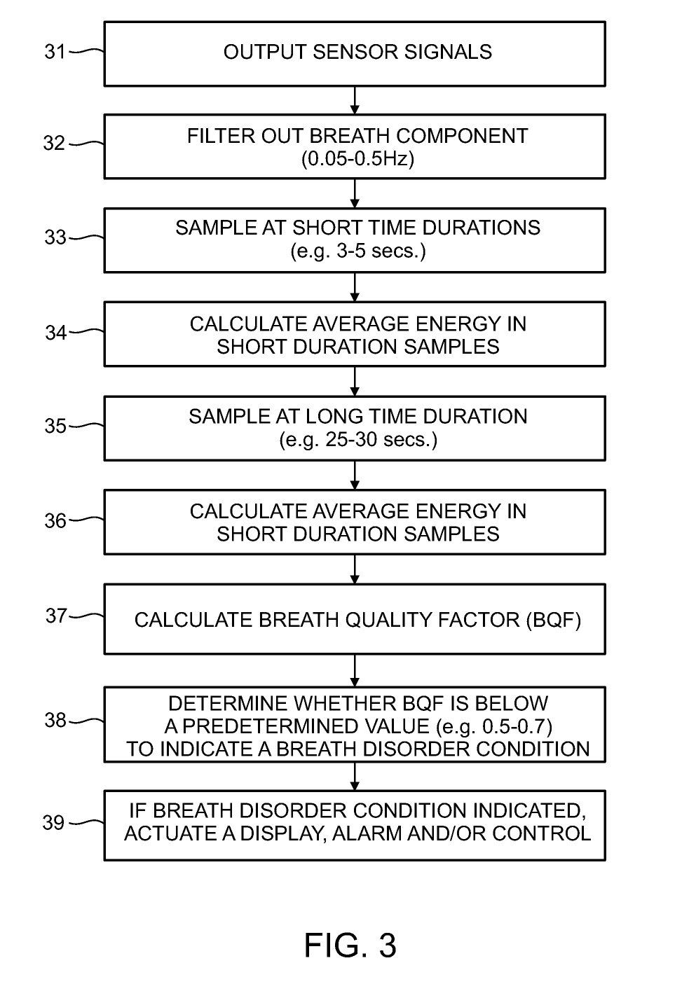

The control system 20 illustrated in FIG. 2 measures the changes in the transit times of the acoustical waves through transmission channel 15, processes the outputs, as described below particularly with respect to the flowchart of FIG. 3, and actuates a display, alarm, and/or control.

Briefly, control system 20 operates by: (a) transmitting from transmitter 13 a cyclically-repeating energy wave through the transmission channel 15 defined with receiver 14; (b) changing the frequency of the transmission while maintaining the number of waves in the loop including the acoustical transmission channel as a whole integer; and (c) utilizing the changes in frequency of the transmission to provide an indication of the deformation of the force applied. Operation (b) includes: detecting a predetermined fiducial point in each cyclically-repeating energy wave received by receiver 14; and continuously changing the frequency of the transmission in accordance with the detected fiducial point of each received energy wave such that the number of energy waves in the loop of the transmission channel is a whole integer.

More particularly, control system 20 illustrated in FIG. 2 operates as follows: Initially, oscillator 21 is energized while switch 22 is closed so as to cause transmitter 13 to transmit a succession of sonic pulses until such pulses are received by receiver 14. Once the pulses are received by receiver 14, switch 22 is opened so that the pulses received by receiver 14 are thereafter used for controlling the transmitter 13.

The sonic signals received by receiver 14 are fed to a comparator 23 via its input 23a. Comparator 23 includes a second input 23b connected to a predetermined bias so as to detect a predetermined fiducial or reference point in the received signal. In the example illustrated, this predetermined fiducial point is the “zero” cross-over point of the received signal; therefore, input 23b of comparator 23 is at a zero bias.

The output of comparator 23 is fed to an amplifier 24, e.g., a monostable oscillator, which is triggered to produce an output signal at each fiducial point (zero cross-over point) in the signals received by receiver 14. The outputs from amplifier 24 are fed via an OR-gate 25 to trigger the transmitter 13 for the next sonic pulse. Since switch 22 is open, transmitter 13 will thus be triggered by each signal received by the receiver 14 to transmit the next sonic pulse in the succession of pulses.

It will thus be seen that the frequency of the output pulses or signals from transmitter 13 will change with a change in the spacing between the transmitter 13 and receiver 14. It will also be seen that the number of wavelengths or pulses in the loop including transmitter 13 and receiver 14 will be a whole integer. This change in frequency by the transmitter 13, while maintaining the number of waves between the transmitter and receiver 14 as a whole integer, enables a precise determination to be made of the distance between the transmitter and receiver, and thereby of the deformation of wall 11.

A summing circuit, including counter 26, counter 27, clock 28 and microprocessor 29, enables the detected frequency difference, and thereby the measurement precision, to be increased by a factor “N”. Thus, the precision of the measurement can be preset, almost without limitation, by the selection of the appropriate frequency, clock rate for clock 28, and summation factor “N” for counter 27.

The output from microprocessor 29 of the control and processor circuit 20 may be used for display, alarm and/or control purposes, as schematically shown at 29a, 29b and 29c.

Further details of the construction and operation of such measuring and processing circuits are described in U.S. Pat. No. 6,621,278 and the above-cited PCT Publication No. WO2008/012820, the contents of which are incorporated herein by reference.

When the apparatus illustrated in FIGS. 1 and 2 is used for sensing breathing disorders in accordance with the present invention, the measured signal is filtered by a microprocessor. A digital (software) filter is used, which eliminates the DC and high-frequency components, to output only the breath components of the signal, in the range of 0.05-0.5 Hz. This filtered output is then passed to the control system 20 which processes the output according to the flowchart illustrated in FIG. 3.

Thus, as seen in FIG. 3, the outputted sensor signals (box 31) are passed through filter 30 to filter out the breath component, preferably 0.05-0.5 Hz (block 32). The output signals are then sampled for short time durations, preferably 3-5 seconds (block 33), and the average energy (E_{S}) of the short time duration samples is calculated (block 34). Similarly, samples of the filtered output are taken during long time durations, preferably 25-30 seconds (block 35), and the average energy (E_{L}), in the long time duration samples, is calculated (block 36).

The short-time duration and long-time duration average energy signals are calculated according to the following equation:

$$E ( k ) = {\sum }_{i - N + 1}^{k} {S}^{2} ( i ) \cdot Δ t$$

Here Δt—signal sampling (about 10 msec);
S(i)—i-th sample of the filtered signal;
k—the number of current sample;
N—the length of sliding window (duration).

The control system then calculates the Breath Quality Factor, BQF, (block 37), according to the following equation:

$$B Q F ( k ) = \frac{{E}_{S} / {N}_{S}}{{E}_{L} / {N}_{L}} ;$$

A determination is then made whether the BQF is below a predetermined value, preferably 0.5-0.7, and if so, this indicates a breath disordered breathing (block 38), and if such a determination is made, the display 29a, alarm 29b, and/or control 29c is actuated.

It will thus be seen that the BQF compares the average powers of the filtered signal in short and short windows or time durations. When breathing is normal, the amplitude of breath is almost a steady one, and therefore the average powers in both windows would be similar such that BQF would be about 1. A breath disorder is accompanied by a change in amplitude. For example, the events of obstructive apnea are accompanied by a decrease of chest movement. In such case, the “short-duration energy” will drop rapidly as well. When BQF is less than a predetermined threshold, preferably about 0.5-0.7, this indicates the occurrence of a breath disorder.

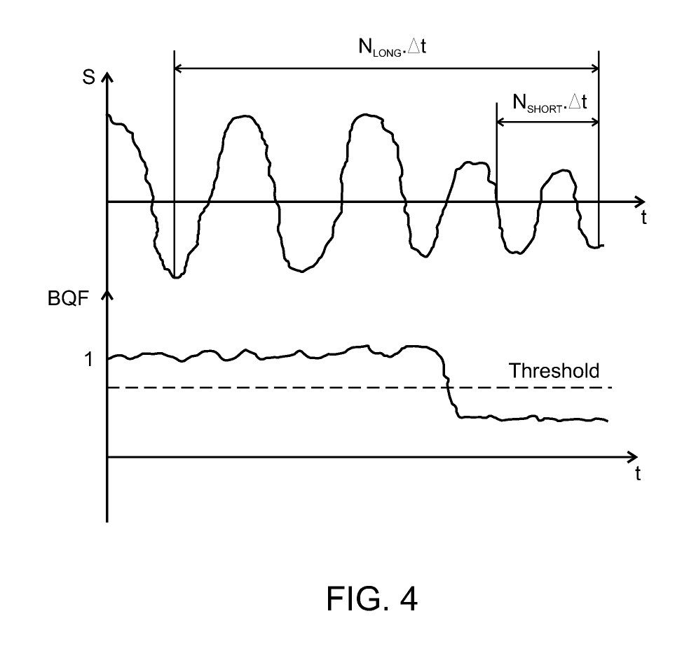

The foregoing is illustrated in the wave forms of FIG. 4.

It has been found that the above method, as illustrated in the flowchart of FIG. 3, is robust, reliable and very simple to implement.

While the invention has been described with respect to one preferred embodiment, it will appreciated that this is set forth merely for purposes of example, and that many other variations, modifications and applications of the invention may be made.

What is claimed is:

1. Apparatus for detecting breathing disorders in a person, comprising:
a sensor constructed to be located on, in, or under a mattress and to sense mechanical vibrations of a body part of the person while lying on the mattress;
and a control system designed to receive the output of said sensor, and to perform the following operations:
(a) filter out the breath components of the sensor output;
(b) sample said breath components during predetermined short time durations (N_{S});
(c) determine the average energy E_{S} in said samples;
(d) sample said breath components during long time durations (N_{L});
(e) determine the average energy (E_{L}) in said latter samples;
(f) calculate a breath quality factor (BQF) by dividing E_{S} by E_{L} (E_{S}/E_{L}); and
(g) actuating a display, alarm, and/or control when the BQF is a predetermined value less than 1.0.

2. The apparatus according to claim 1, wherein in operation (a), the breath components filtered out are of a frequency of 0.05-0.5 Hz.

3. The apparatus according to claim 1, wherein in operation (b), said predetermined short time durations are 3-5 seconds, and in operation (d) said predetermined long time durations are 25-30 seconds.

4. The apparatus according to claim 1, wherein in operations (c) and (e), the average energy of said predetermined short time durations (E_{S}) and said predetermined long time durations (E_{L}) are calculated as follows:
$$E ( k ) = {\sum }_{i - N + 1}^{k} {S}^{2} ( i ) \cdot Δ t$$
Here Δt—signal sampling (about 10 msec);
S(i)—th sample of the filtered signal;
k—the number of current sample;
N—the length of sliding window (duration).

5. The apparatus according to claim 4, wherein in operation (f), the breath quality factor is calculated as follows:
$$B Q F ( k ) = \frac{{E}_{S} / {N}_{S}}{{E}_{L} / {N}_{L}}$$

6. The apparatus according to claim 4, wherein in operation (g), the display, alarm and/or control is actuated when the breath quality factor is between 0.5-0.7.

7. The apparatus according to claim 1, wherein said sensor is an acoustical sensor.

8. The apparatus according to claim 7, wherein said acoustical sensor includes:
an acoustical transmitter;
an acoustical receiver spaced from said acoustical transmitter to define an acoustical transmission channel therebetween adapted to be located, with respect to the person, such that said mechanical vibrations of the person's body part change the length of said acoustical transmission channel;
and a measuring circuit for measuring the transit time of an acoustical signal transmitted from said acoustical transmitter to said acoustical receiver.

9. A method for detecting breathing disorders in a person, comprising:
sensing mechanical vibrations of a body part of a person while lying on a mattress;
converting said mechanical vibrations to electrical signals;
and processing said electrical signals by the following operations:
(a) filtering out the breath components of the sensor output;
(b) sampling said breath components during predetermined short time durations (N_{S});
(c) determining the average energy E_{S} in said samples;
(d) sampling said breath components during long time durations (N_{L});
(e) determining the average energy (E_{L}) in said latter samples;
(f) calculating a breath quality factor (BQF) by dividing E_{S} by E_{L} (E_{S}/E_{L}); and
(g) actuating a display, alarm, and/or control when the BQF is a predetermined value less than 1.0.

10. The method according to claim 9, wherein in operation (a), the breath components filtered out are of a frequency of 0.05-0.5 Hz.

11. The method according to claim 9, wherein in operation (b), said predetermined short time durations are 3-5 seconds, and in operation (d) said predetermined long time durations are 25-30 seconds.

12. The method according to claim 9, wherein in operations (c) and (e), the average energy of said predetermined short time durations (E_{S}) and said predetermined long time durations (E_{L}) are calculated as follows:
$$E ( k ) = {\sum }_{i - N + 1}^{k} {S}^{2} ( i ) \cdot Δ t$$
Here Δt—signal sampling (about 10 msec);
S(i)—i-th sample of the filtered signal;
k—the number of current sample;
N—the length of sliding window (duration).

13. The method according to claim 12, wherein in operation (f), the breath quality factor is calculated as follows:
$$B Q F ( k ) = \frac{{E}_{S} / {N}_{S}}{{E}_{L} / {N}_{L}}$$

14. The method according to claim 13, wherein in operation (g), the display, alarm and/or control is actuated when the breath quality factor is between 0.5-0.7.

15. The method according to claim 9, wherein said sensor is an acoustical sensor.

16. The method according to claim 15, wherein said acoustical sensor includes:
an acoustical transmitter;
an acoustical receiver spaced from said acoustical transmitter to define an acoustical transmission channel therebetween adapted to be located, with respect to the person, such that said mechanical vibrations of the person's body part change the length of said acoustical transmission channel;
and a measuring circuit for measuring the transit time of an acoustical signal transmitted from said acoustical transmitter to said acoustical receiver.
